(19) United States
(12) Patent Application Publication
(10) Pub. No.: US 2013/0252339 A1
(43) Pub. Date: Sep. 26, 2013

(54) MICRORNA-INDUCED ES-LIKE CELLS AND USES THEREOF

(71) Applicant: UNIVERSITY OF SOUTHERN CALIFORNIA, Los Angeles, CA (US)

(72) Inventors: Shi-Lung LIN, Arcadia, CA (US); Shao-Yao YING, San Marino, CA (US)

(73) Assignee: UNIVERSITY OF SOUTHERN CALIFORNIA, Los Angeles, CA (US)

(21) Appl. No.: 13/776,464

(22) Filed: Feb. 25, 2013

Related U.S. Application Data
(63) Continuation of application No. 13/541,398, filed on Jul. 3, 2012, now abandoned.
Application No. 13/541,398 is a continuation of application No. 13/296,068, filed on Nov. 14, 2011, now abandoned.
Application No. 13/296,068 is a continuation of application No. 12/808,647, now abandoned.
(60) Provisional application No. 61/007,867, filed on Dec. 17, 2007.
(60) Provisional application No. 61/060,416, filed on Jun. 10, 2008.
(60) Provisional application No. 61/074,481, filed on Jun. 20, 2008.

(51) Int. Cl. C12N 15/113

(52) U.S. Cl. CPC C12N 15/113
USPC 435/455; 435/320.1; 435/325; 800/13

(57) ABSTRACT

The invention relates to isolated nucleic acids comprising mir-302 genes. Also disclosed are expression vectors, host cells, and transgenic animals containing the nucleic acids, and use of the nucleic acids to generate ES-like cells.

### RELATED APPLICATIONS

This application claims priority to U.S. Provisional Application Ser. Nos. 61/007,867, filed on Dec. 17, 2007, 61/060,416, filed on Jun 10, 2008, and 61/074,481, filed on Jun. 20, 2008, the contents of which are incorporated herein by reference in their entirety.

### FIELD OF THE INVENTION

The present invention relates in general to microRNAs. More specifically, the invention provides isolated nucleic acids comprising mir-302 genes, and expression vectors, host cells, and transgenic animals containing such nucleic acids. The invention further provides methods of generating ES-like cells using microRNAs.

### BACKGROUND OF THE INVENTION

The concept of cancer stent cells indicates that transformed stem cells within a tumor are able to self-renew and differentiate into a heterogeneous tumor population (Reya et al., 2001). However, there is no clear mechanism underlying such stem-cancer cell transformation or vice versa. In the clinic, it is very frequent to observe that cancer progression is generally associated with the poor differentiation (high grade) of human tumor cells. Recent findings have also shown that poorly differentiated tumors preferentially over-express genes normally enriched in human embryonic stem (ES) cells, such as targets of Oct3/4, Sox2 and Nanog transcription factors; nevertheless, the concurrent expression of these transcription factors themselves is not often detected in the poorly differentiated tumors (Ben-Porath et al., 2008). It is conceivable that a different set of transcriptional regulators in place of the roles of Oct3/4, Sox2 and Nanog may function in poorly differentiated tumor cells to promote their “stemness” signatures. Therefore, finding this way how stem cells hurdle these cancer-related transcriptional regulators may shed light on breakthroughs in both cancer therapy and stem cell generation.

The mir-302 family (mir302s) generally consists of four highly homologous microRNA (miRNA) members, which are transcribed together as a non-coding RNA cluster containing mir-302b, mir-302c, mir-302a, mir-302d, and mir-367 from a 5′ to 3′ direction (Sub. et al., 2004). They are expressed most abundantly in slow-growing human ES cells and the expression quickly decreases alter cell differentiation and proliferation (Suh et al., 2004). Given that miRNAs are characterized as small inhibitory RNAs capable of suppressing the translation of target genes with high complementarity (Bartel, D. P., 2004), mir-302s is a likely candidate of zygotic inhibitors to prevent premature cell differentiation during early embryonic development. As shown in the miRBase::Sequences program at the website of microrna.sanger.ac.uk, mir-302s can target over 445 human genes and most of these targets are developmental signals involving the initiation and/or facilitation of lineage-specific cell differentiation during early human embryogenesis.

### SUMMARY OF THE INVENTION

The present invention is based, at least in part, upon the unexpected discovery that mir-302s reprogram human skin cancer cells into a pluripotent ES-cell-like state.

Accordingly, in one aspect, the invention features an isolated nucleic acid comprising one or more mir (microRNA)-302 genes operably linked to a regulatory sequence. The regulatory sequence controls the expression of the mir-302 genes.

In another aspect, the invention features an isolated nucleic acid comprising a regulatory sequence operably linked to a recombinant sequence encoding a contiguous transcript. The regulatory sequence controls the transcription of the recombinant sequence. The recombinant sequence comprises a first gene including at least two exons flanking one intron. The intron comprises one or more mir-302s. The intron is spliced out of the contiguous transcript of the recombinant sequence to allow the mir-302s to interact with their targets in a cell.

A mir-302 gene may be the mir-302a, mir-302b, mir302c, or mir-302d gene. Likewise, a mir-302 may be mir-302a, mir-302b, mir-302c, or mir-302d. In some embodiments, a nucleic acid of the invention is transcribed by a type II RNA polymerase. In some embodiments, an intron of the invention is spliced out of the contiguous transcript of the recombinant sequence by a spliceosome. Exemplary first genes include but are not limited to the RGFP (red fluorescent HcRed1 chromoprotein) gene or a fragment thereof.

The invention also provides an expression vector, a host cell, and a transgenic animal comprising a nucleic acid of the invention.

In addition, the invention provides a method of generating ES (embryonic stem)-like cells. The method comprises contacting non-ES-like cells with a nucleic acid of the invention, thereby transforming the non-ES-like cells into ES-like cells. In some embodiments, a method of the invention further comprises inducing the ES-like cells to differentiate into tissue cell types.

The non-ES-like cells may be cancer cells such as Colo or PC3 cells. Such cells, when transformed into ES-like cells, may form a teratoma-like primordial tissue structure, fibroblasts, chondrocytes, spermatogonia-like primordial cells, or neuronal cells.

Unless otherwise defined, all technical and scientific terms used herein have the same meaning as commonly understood by one of ordinary skill in the art to which this invention pertains. In case of conflict, the present document, including definitions, will control. The materials, methods, and examples disclosed herein are illustrative only and not intended to be limiting. Other features, objects, and advantages of the invention will be apparent from the description and the accompanying drawings, and from the claims.

### BRIEF DESCRIPTION OF THE FIGURES

FIG. 1. Strategy for generating transgenic mir-302s-expressing mirPS cell lines, using retrovirus-based pLNCX2-rT-SpRNAi vector transfection. A retroviral delivery approach was used to integrate a cytomegalovirus (CMV) promoter-driven SpRNAi-RGFP transgene into the tested cell genomes for steady expression of a manually redesigned mir-302 pre-miRNA cluster (mir-302s). Mir-302s was placed in the intron of the SpRNAi-RGFP transgene and generated as a part of the transgene transcript RNA (pre-mRNA), containing RGFP protein-coding exons and non-coding introns. The introns were spliced out of pre-mRNA and further excised into small miRNA-like mir-302 molecules capable of triggering targeted gene silencing, while the RGFP exons were ligated together to form a mature mRNA for synthesis of a red fluorescent marker protein, RGFP. The presence of RGFP served as an indicator for the expression and processing of mir-302s.

FIG. 2. Reprogramming of human cancerous Colo and PC3 cells into ES-like. mirPS cells with retrovirus-mediated transfection of mir-302s. (A) Structure of a mir-302s-expressing SpRNAi-RGFP transgene located in the XhoI/AflII cloning site of a cytomegalovirus (CMV) promoter-driven pLNCX2 retroviral vector (Clontech), namely pLNCX2-rT-SpRNAi. (B) Construct of the mir-302 pre-miRNA cluster (mir-302s), which was inserted in the intron region of the SpRNAi-RGFP transgene. (C) Selection of mirPS cells using FACS flow cytometry sorting with antibodies to RGFP and Oct3/4. (D) Changes of morphologies and cell division rates in mirPS cells. The first (left) and second (right) peaks of the DNA-density flow cytometry charts represented the levels of resting G0/G1 and mitotic M phase cell populations in the entire tested cell population, respectively. The mir-gfp miRNA shared no homology to human genes. (E) Loss of migration ability in mirPS-PC3 cells as compared to its original PC3 cells. (F) Formation of embryoid bodies derived from mirPS cells and their differentiation into neuron-like primordial cells with Tuj1 and ABCA2 marker expression.

FIG. 3. Teratoma-like primordial tissues derived from the mirPS EB implants in the uterus or peritoneal cavity of female pseudopregnant immunocompromised SCID-beige mice. These differentiated tissues included all three embryonic germ layers—ectoderm, mesoderm and endoderm, as determined by their distinct cell morphologies after hematoxylin and eosin (H&E) staining. Photographs were taken with the Nikon TE2000 microscopic system at 200× magnification.

FIG. 4. Correlation among mir-302 transaction, ES marker expression and genomic DNA demethylation in mirPS cells. (A) Microarray analyses of miRNA expression, revealing that all mir-302 familial members (mir-302s) were highly expressed in the mirPS-Colo rather than original Colo cells (n=3, p<0.01). (B) Western blot analyses, showing that mirPS cells expressed high levels of human ES cell markers, including Oct3/4, SSEA-3, SSEA-4, Sox2 and Nanog, but not oncogenic Klf4 (n=4, p<0.01). (C) HpaII cleavage showing the loss of global CpG methylation at a genome-wide scale in mirPS cells. (D) Bisulfite modification of unmethylated ACGT into AUGT sites in the 9,400-bp regulatory region of the Oct3/4 promoter, showing an increase of unmethylated ACTG (AUCT) sites in mirPS cells. (E) Bisulfite DNA sequencing, showing the detailed methylation maps flanking the transcription initiation site, of the Oct3/4 promoter. Black and white circles indicate the methylated and unmethylated cytosine sites, respectively.

FIG. 5. Genome-wide gene expression analyses among Colo, mirPS-Colo and human ES WA01-H1 (H1) and WA09-H9 (H9) cells. (A) Comparison of altered gene expression patterns using Human genome GeneChip U133A&B and plus 2.0 arrays (Affymetrix), showing high similarity between mirPS-Colo and H1 (89%) as well as H9 (86%), but not original Colo (53%) cells. (B)-(E) Functional clustering of microarray-identified differentially expressed genes, demonstrating that a significant increase of ES cell markers (B) and a marked decrease of melanoma oncogenes (C), developmental signals (D), and mir-302s-targeted cell proliferation and DNA methylation genes (E) were detected in mirPS cells, which highly resembled those in H1 and H9 cells (n=4, p<0.01). Any signal showing above the level 23,000 of total 36,535 (in red) was considered to be a positive call in the gene expression list.

FIG. 6. Pluripotency of mirPS cells. Treatments of DHT, TGF-β1 and BMP4, respectively from top to bottom, induced the mirPS-Colo cell differentiation into spermatogonia-like (A-E), fibroblast-like (F-J) and chondrocyte-like (K-O) primordial cells, in immunocompromised mice ex vivo. The use of immunocompromised nude mice was to provide an in vivo environment mimicking transplantation therapy. Microscopic photographs shown from left to right indicated hematoxylin staining with differential interference contrast (A, F, K), bright field labeled with transgenic mir-302 marker RGFP (red) (B, G, L), immunostaining of the first tissue marker labeled with 4,6-diamidino-2-phenylindole (blue: DAPI) (C, H, M), immunostaining of the second tissue marker labeled with fluorescein (green EGFP) (D, I, N), and merge of all three fluorescent markers (E, J, O). Small windows in the RGFP-bright fields showed the morphologies of differentiated mirPS cells at high magnification (600 ×).

FIG. 7. List of differentially enriched miRNAs in mirPS-Colo cells. The expression of mir-302 familial members, including mir-302a, mir-302b, mir-302 c and mir-302d, were all highly elevated in mirPS-Colo (sample B) as compared to the original Colo (sampler A) cells. Concurrently, one of the reverse mir-302, mir-302a*, was also markedly expressed.

FIG. 8. Transgenic integration of the mir-302s-expressing SpRNAi-RGFP transgene in mirPS cells. (A) Quantitative PCR analyses of the genomic DNAs isolated from mirPS cells, showing that all tested mirPS cells carried only one or two copies of the transgene, whereas no transgene was detected in the original Colo and PC3 cells. (B) Fluorescent in situ hybridization (FISH) detection of genomic transgene insertion. Approximately 75% of mirPSPC3 and 17% of mirPS-Colo cells contained one transgene insert in their genomes, while the others contained two inserts, but no more than three. Many of these two inserts were concomitantly placed to each other, an event frequently observed in high-titer retroviral infection. Such restricted transgene insertion indicates that the mir-302 expression level may affect the survival of the mirPS cells.

### DETAILED DESCRIPTION OF THE INVENTION

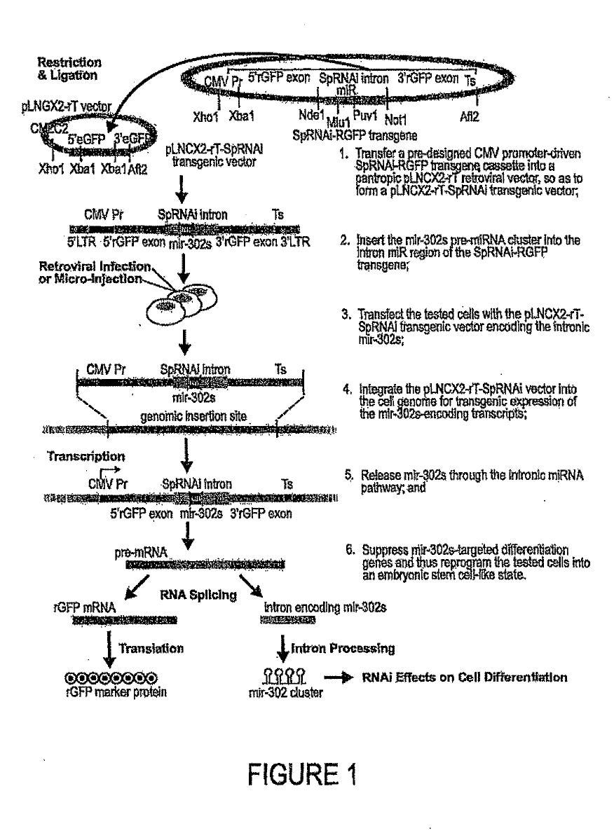

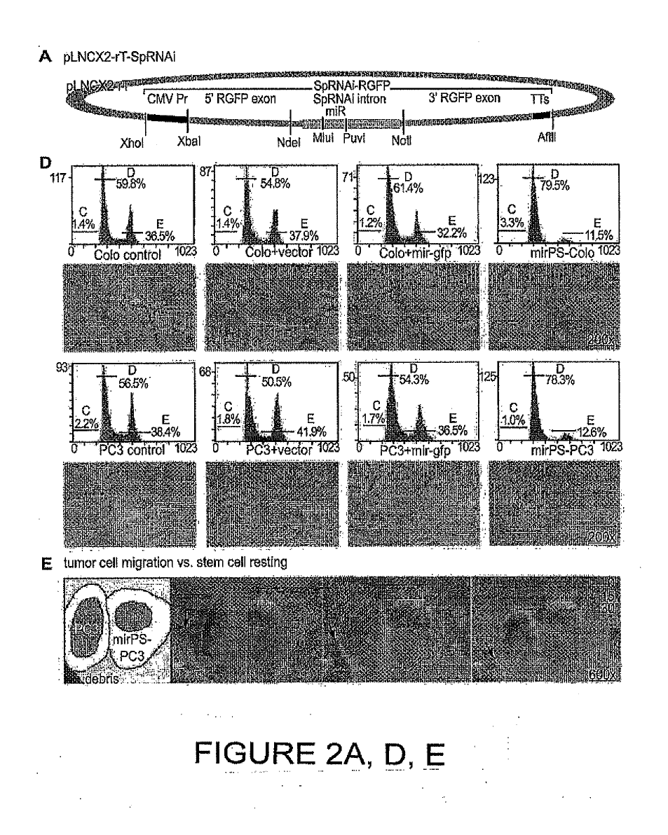

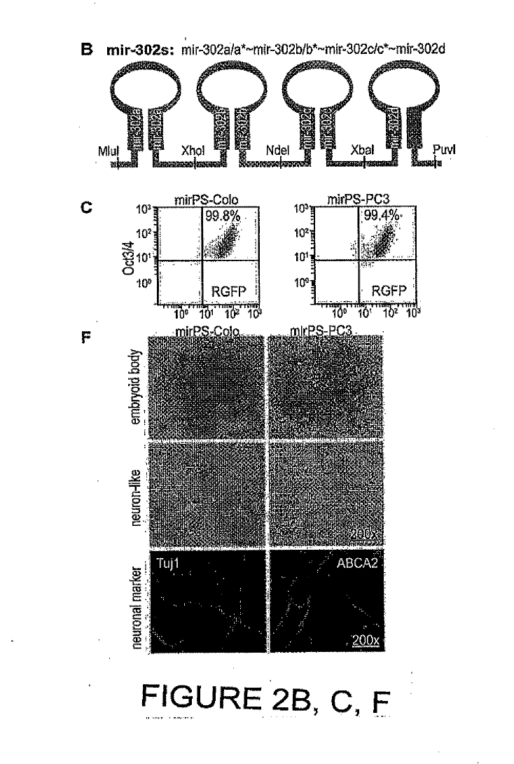

The present invention relates to the utilities of microRNAs in generating ES-like cells. As described in detail below, to test the function of mir-302s, a retroviral Pol-II-based intronic miRNA expression system was developed, namely pLNCX2-rT-SpRNAi (FIG. 1), and successfully used to generate several transgenic miRNA-expressing cell lines and animals (Lin and Ying, 2006; Lin et al., 2006). The same transgenic approach has also been used to generate gene-knockout mice for human disease research (Xia et al., 2006). Intronic miRNA expression is a prevalent event in mammals because approximately 50% of mammalian miRNAs are encoded within the introns of protein-coding genes (Rodriguez et al., 2004). These miRNAs are transcribed by type II RNA polymerases. (Pol II) and excised by spliceosomes and other RNaseIII endonucleases to form mature miRNAs (Lin et al., 2003; Danin-Kreiselman et al., 2008). However, Drosha may not be required for this process (Ruby et al., 2007). The composition of this mir-302s-expressing pLNCX2-rT-SpRNAi vector is shown in FIG. 2A. Using this vector-based transaction strategy, two mir-302s-expressing mirPS cell lines were generated, namely mirPS-Colo and mirPSPC3, derived from human melanoma Colo and prostate cancer PC3 cellar, respectively, and their ES-like cell renewal and pluripotent properties confirmed.

Accordingly, the invention provides an isolated nucleic acid comprising one or more mir-302 genes operably linked to a regulatory sequence, wherein the regulatory sequence controls the expression of the mir-302 genes.

The term “isolated nucleic acid” includes nucleic acid molecules that are separated from other nucleic acid molecules that are present in the natural source of the nucleic acid. For example, with respect to genomic DNA, the term “isolated” includes nucleic acid molecules that are separated from the chromosome with which the genomic DNA is naturally associated. Preferably, an “isolated” nucleic acid is free of sequences that naturally flank the nucleic acid (i.e., sequences located at the 5′ and/or 3′ ends of the nucleic acid) in the genomic DNA of the organism from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

mir-302 genes are known in the art. The mir-302 family (mir-302s) generally consists of four highly homologous members, mir-302a, mir-302b, mir-302c, mir-302d. For example, the human mir-302 cluster (sometimes called the human cluster miR302-367) contains eight different miRNAs cotranscribed in a polycistronic way (Suh et al., 2004): miR302a, miR302a*, miR302b, miR302b*, miR302c, miR302c*, miR302d, and miR367. The chromosomal locations of human family mir-302s are: hsa-mir-302a, MI0000738; hsa-mir-302b, MI0000772; hsa-mir-302c, MI0000773; hsa-mir-302d, MI0000774.

Genes and RNAs described herein can be replaced by their functionally equivalent fragments or homologs (e.g., with at least 50%, 60%, 70%, 80%, or 90% sequence homology). In particular, mir-302a, mir-302b, mir-302c, and mir-302d genes and RNAs described herein may be replaced with other genes and RNAs with similar functions such as mir-302a*, mir-302b*, mir-302c*, mir-367, mir-93, mir-371, mir-372, mir-373, mir-520, and the like.

The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences.

Another isolated nucleic acid of the invention comprises a regulatory sequence operably linked to a recombinant sequence encoding a contiguous transcript. The regulatory sequence controls the transcription of the recombinant sequence. The recombinant sequence comprises a first gene encoding at least two exons flanking one intron. The intron comprises one or more mir-302s. The intron is spliced out of the contiguous transcript of the recombinant sequence to allow the mir-302s to interact with their targets in a cell.

As used herein, a “recombinant” sequence refers to a sequence that does not occur in nature.

Preferably, the nucleic acid is transcribed by a type II RNA polymerase, and the intron is spliced out of the contiguous transcript of the recombinant sequence by a spliceosome. Regulatory sequences such as promoters for type II RNA polymerases are generally known in the art. See, for example, Smale and Kadonaga (2008) Annu Rev Biochem 72:449-479. The cis and trans elements-required for spliceosomal splicing are also known to a skilled artisan. See, for example, Lewin B., Genes, Seventh Edition, Oxford University press, page 689, 2000. Therefore, one skilled in the art may construct a nucleic acid of the invention using recombinant DNA techniques or chemical synthesis.

To monitor the transcription of the recombinant sequence and subsequent processing of the transcript, a detectable marker gene may be used as the first gene. For example, as described in detail below, when the RGFP gene is employed as the first gene, the presence of the RGFP protein serves as an indicator for the transcription of the recombinant sequence and the processing of the transcript.

A nucleic acid of the invention can be included in a vector, preferably an expression vector. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a nucleic acid of the invention in a form suitable for the expression of the nucleic acid in a host cell. The design of the expression vector can depend on such factors as the choice of the host cell to be transformed, the level of mir-302 gene expression desired, and the like. The expression vectors of the invention can be introduced into host cells to thereby produce mir-302s.

The expression vectors of the invention can be designed for the expression of the mir-302 genes in a variety of cells such as insect cells (e.g., using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the expression vector can be transcribed in vitro, for example using T7 promoter regulatory sequences and T7 polymerase.

When used in mammalian cells, the expression vector's control functions are often provided by viral regulatory elements. For example, commonly used promoters are derived from polyoma, Adenovirus 2, cytomegalovirus and Simian Virus 40.

In some embodiments, an expression vector of the invention is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,816 and European Application Publication No. 264,166). Developmentally regulated promoters are also encompassed, including for example, the murine hox promoters (Kessel and Gruss (1990) Science 249:374-379) and the α-fetoprotein promoter (Campes and Tilghman (1989) Genes Dev. 3:537-546).

The invention further provides a host cell that includes a nucleic acid of the invention. The nucleic acid may be within an expression vector or homologously recombine into a specific site of the host cell's genome. The terms “host cell” refers not only to the particular subject cell but to the progeny or potential progeny of such a cell.

A nucleic acid or vector of the invention can be introduced into host cells via conventional transformation or transfection techniques. As used herein, the terms “transformation” and “transfection” are intended to refer to a variety of art-recognized techniques for introducing a foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) mir-302s. Accordingly, the invention further provides methods for producing mir-302s using the host cells of the invention. In one embodiment, the method includes culturing the host cells of the invention in a suitable medium such that mir-302s are produced.

The invention additionally features non-human transgenic animals containing a nucleic acid of the invention. Such animals are useful for studying the function and/or activity of mir-302s. As used herein, a “transgenic animal” is a non-human animal, preferably a mammal, more preferably a rodent such as a rat or mouse, in which one or more of the cells of the animal include a transgene containing a nucleic acid of the invention. Other examples of transgenic animals include non-human primates, sheep, dogs, cows, goats, chickens, amphibians, and the like. A transgene is an exogenous DNA, which preferably is integrated into the genome of the cells of a transgenic animal. A transgene can direct the expression of an encoded gene product in one or more cell types or tissues of the transgenic animal.

Intronic sequences and polyadenylation signals can also be included in the transgene to increase the efficiency of the expression of the transgene. A tissue-specific regulatory sequence can be operably linked to a transgene of the invention to direct the expression of mir-302s to particular cells. A transgenic founder animal can be identified based upon the presence of a transgene in its genome and/or expression of mir-302s in tissues or cells of the animal. A transgenic founder animal can then be used to breed additional animals carrying the transgene. Moreover, transgenic animals carrying a transgene can further be bred to other transgenic animals carrying other transgenes.

The invention also includes a population of cells from a transgenic animal, as discussed herein.

A method of generating ES-like cells is within the invention. The method involves contacting non-ES-like cells with a nucleic acid of the invention, thereby transforming the non-ES-like cells into ES-like cells. This method may be practiced in vivo, in vitro, or ex vivo.

The term “ES-like cells” refers to cells derived from adult or mature, non-pluripotent cells but have many or all of the characteristics of embryonic stem cells. ES-like cells may be identified using protocols well known in the art. For example, as described in detail below, ES-like cells may be identified using antibodies to ES markers such as Oct3/4, SSEA-3, SSEA-4, Sox2 and Nanog, by detecting a slow rate of cell cycle, changes in cell morphology, loss of ability to migrate, genomic demethylation, or inhibition of cell cycle checkpoint genes (e.g., CDK2 and cyclin D1 and D2) and DNA methylation facilitator genes (e.g., MECP2 and MECP1 component p66), or by detecting the pluripotency of the cells.

When a nucleic acid of the invention is introduced into non-ES-like cells, mir-302s are produced from the nucleic acid and interact with their targets in the cells. Most of these target genes are developmental signals involving the initiation and/or facilitation of lineage-specific cell differentiation during early embryogenesis. Thus, mir-302s are key factors essential for ES cell maintenance.

As described in detail below, cancer cells can be reprogrammed by mir-302s into a more ES-like state. Accordingly, a nucleic acid of the invention may be used in a cancer therapy. A treatment method of the invention involves administering an effective amount of a nucleic acid of the invention to a subject suffering from cancer.

As used herein, a “subject” refers to a human or animal, including all mammals such as primates (particularly higher primates), sheep, dog, rodents (e.g., mouse or rat), guinea pig, goat, pig, cat, rabbit, and cow. In a preferred embodiment, the subject is a human. In another embodiment, the subject is an experimental animal or animal suitable as a disease model.

A subject to be treated may be identified in the judgment of the subject or a health pare professional, which can be subjective (e.g., opinion) or objective (e.g., reached by detecting a cancer marker in the subject).

A “treatment” is defined as administration of a substance to a subject with the purpose to cure, alleviate, relieve, remedy, prevent, or ameliorate a disorder, symptoms of the disorder, a disease state secondary to the disorder, or predisposition toward the disorder.

An “effective amount” is an amount of a compound that is capable of producing a medically desirable result in a treated subject. The medically desirable result may be objective (i.e., measurable, by some test or marker) or subjective (i.e., subject gives an indication of or feels an effect).

For treatment of cancer, a compound is preferably delivered directly to tumor cells, e.g., to a tumor or a tumor bed following surgical excision of the tumor, in order to treat any remaining tumor cells.

Nucleic acids can be delivered to target cells by, for example, the use of polymeric, biodegradable microparticle or microcapsule devices known in the art. Another way to achieve uptake of nucleic acids is to use liposomes, prepared by standard methods. The nucleic acids can be incorporated alone into these delivery vehicles or co-incorporated with tissue-specific or tumor-specific antibodies. Alternatively, one can prepare a molecular conjugate composed of a nucleic acid attached to poly-L-lysine by electrostatic or covalent forces. Poly-L-lysine binds to a ligand that can bind to a receptor on target cells. “Naked DNA” (i.e., without a delivery vehicle) can also be delivered to an intramuscular, intradermal, or subcutaneous site. Generally, preferred dosage for administration of nucleic acids is from approximately 10⁶ to 10¹² copies of the nucleic acid molecule.

A nucleic acid of the invention can be incorporated into pharmaceutical compositions. Such compositions typically include the therapeutic compounds and pharmaceutically acceptable carriers. “Pharmaceutically acceptable carriers” include solvents, dispersion media, coatings, antibacterial and antifungal agents, isotonic and absorption delaying agents, and the like, compatible with pharmaceutical administration.

A pharmaceutical composition is formulated to be compatible with its intended route of administration. See, e.g., U.S. Pat. No. 6,756,196. Examples of routes of administration include parenteral, e.g., intravenous, intradermal, subcutaneous, oral (e.g., inhalation), transdermal (topical), transmucosal, and rectal administration.

It is advantageous to formulate oral or parenteral compositions in dosage unit form for ease of administration and uniformity of dosage. “Dosage unit form,” as used herein, refers to physically discrete units suited as unitary dosages for the subject to be treated, each unit containing a predetermined quantity of an active compound calculated to produce the desired therapeutic effect in association with the required pharmaceutical carrier.

The dosage required for treating a subject depends on the choice of the route of administration, the nature of the formulation, the nature of the subject's illness, the subject's size, weight, surface area, age, and sex, other drugs being administered, and the judgment of the attending physician. Suitable dosages are in the range of 0.01-100.0 mg/kg. Wide variations in the needed dosage are to be expected in view of the variety of compounds available and the different efficiencies of various routes of administration. For example, oral administration would, be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

Moreover, a method of the invention may further comprise inducing the ES-like cells to differentiate into tissue cell types. Through in vitro manipulations with different factors and/or hormones, the ES-like cells can differentiate into the three embryonic germ layers (ectoderm, mesoderm and definitive endoderm)—the founders of all adult tissues. Absent any treatment, xenograft implantation of embryoid bodies derived from the ES-like cells into an animal or human can form various tissue structures. For example, as described in detail below, after in vitro treatments of various growth factors and/or hormones, the mirPS-Colo cells differentiate into several tissue cell types ex vivo, including fibroblasts, chondrocytes and spermatogonia-like primordial cells. Xenograft implantation of the mirPS-Colo-derived embryoid bodies into the uterus or peritoneal cavity of female pseudopregnant immunocompromised SCID-beige mice forms teratoma-like primordial tissue structures.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

EXAMPLE I

### Mir-302 Reprograms Human Skin Cancer Cells Into a Pluripotent ES-Cell-Like State

### Abstract

Stem cell renewal differs from cancer cell growth in its highly self-regulated cell division pattern. The mir-302 microRNA family (mir-302s) is expressed most abundantly in slow-growing human embryonic stem (ES) cells, and the expression quickly decreases after cell differentiation and proliferation. Therefore, mir-302s were investigated as the key factors essential for maintenance of ES cell renewal and pluripotency in this study. The Pol II-based intronic microRNA (miRNA) expression system was used to transgenically transfect the mir-302s into several human cancer cell lines. The mir-302-transfected cells, namely miRNA-induced pluripotent stem (mirPS) cells, not only expressed many key ES cell markers, such as Oct3/4, SSEA-3, SSEA-4, Sox2 and Nanog, but also had a highly demethylated genome similar to a reprogrammed zygotic genome. Microarray analyses further revealed that genome-wide gene expression patterns between the mirPS and human ES H1 and H9 cells shared over 86% similarity. Using molecular guidance in vitro, these mirPS cells could differentiate into distinct tissue cell types, such as neuron-, chondrocyte-, fibroblast- and spermatogonia-like primordial cells. Based on these findings, we conclude, that mir-302s not only function to reprogram cancer cells into an ES-like pluripotent state, but also to maintain this state under a feeder-free cultural condition, which offers a great opportunity for therapeutic intervention.

### RESULTS

### Generation of Human ES-Like Mir-302-Induced Pluripotent Stem (mirPS) Cell Lines and Embryoid Bodies

After the pLNCX2-rT-SpRNAi retroviral transfection with a pre-designed mir-302 pre-miRNA cluster transgene (FIG. 2B), approximately 95%-98% of the transfected cells underwent apoptosis with the remaining 2%-5% of the cells transformed into ES-like mirPS cells. The transfection rates of mir-302s info Colo and PC3 cells were 99.8% and 99.4%, respectively, as determined by FACS flow cytometry sorting with mir-302 maker RGFP and ES marker Oct3/4 antibodies (FIG. 2C). These mirPS cells could grow in either DMEM/F12 of RPMI 1640/B27 medium supplemented with 10% charcoal-stripped FBS, 4 mM L-glutamine, 1 mM sodium pyruvate, 5 ng/ml activin, 5 ng/ml noggin, 3 ng/ml bFGF and an equal mixture of 0.5 μM Y27632 and 0.5 μM GSK-3 inhibitor XV, at 37° C. under 5% CO₂. Under this feeder-free cultural condition, the average cell cycle of the mirPS cells was about 20-24 hours, indicating a very slow cell renewal rate as compared to their cancerous counterparts. The flow cytometry analysis comparing DNA content to cell cycle stages showed a greater than 67% reduction in the mirPS mitotic cell population (FIG. 2D). The mitotic cell population (M phase) was decreased from 36.5% to 11.5% in mirPS-Colo and from 38.4% to 12.6% in mirPS-PC3 cells, whereas no change was found in the control cells transfected with either an empty pLNCX2-rT-SpRNAi vector (cell+vector) or a vector encoding an off-target mir-gfp pre-miRNA construct (cell+mirgfp). Accordingly, the mirPS cell morphology (lower panels) was changed from a spindle- or asterisk-like form to a more rounded shape, indicating that the mirPS cells may have lost their ability to migrate. As shown in FIG. 2E, metastatic PC3 cells quickly migrated over time, whereas mirPS-PC3 cells remained stationary. Thus, such transgenic mir-302s expression is sufficient to transform human cancer cells into a more ES-like cell morphology and rate of cell division, indicating a very beneficial use in cancer therapy.

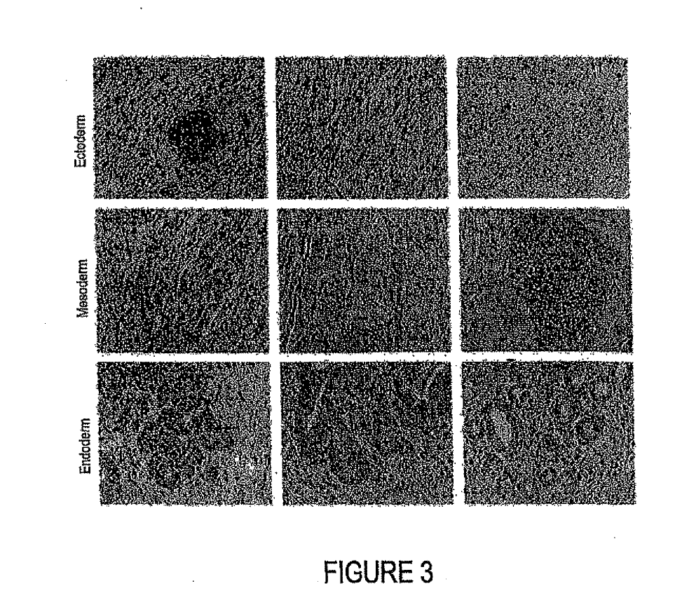

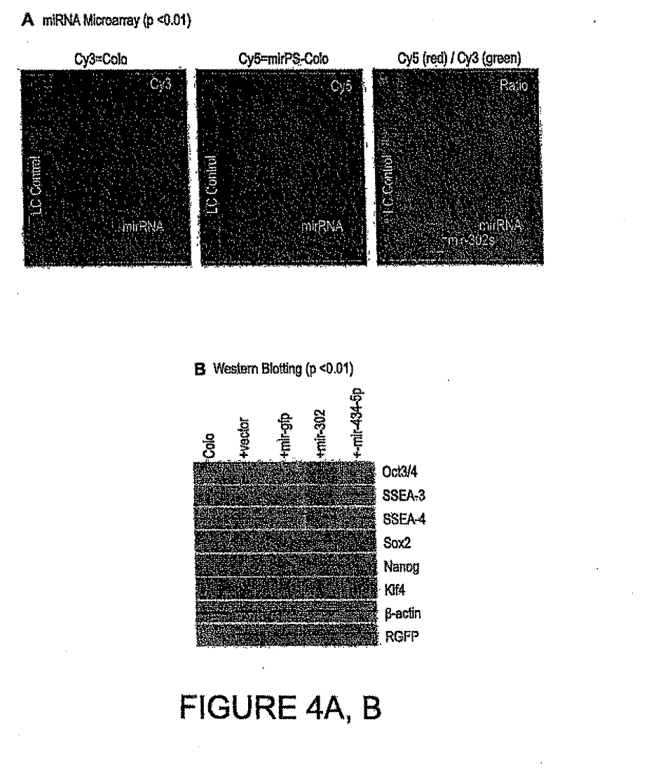

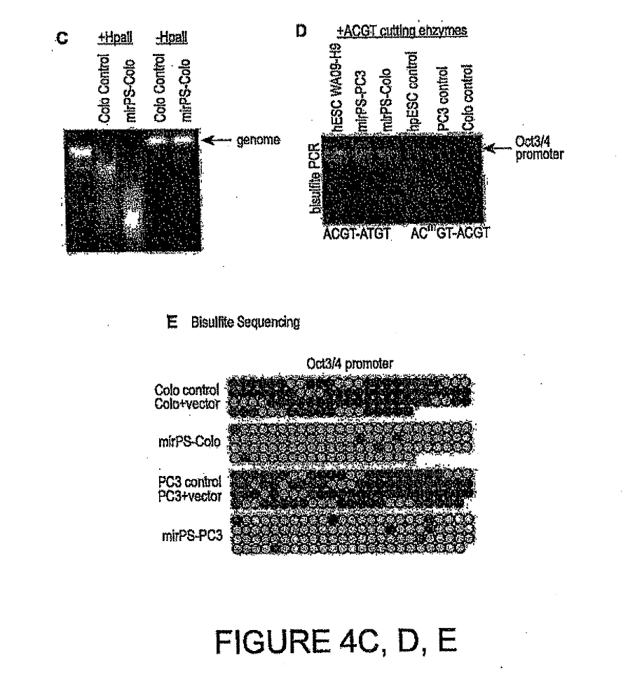

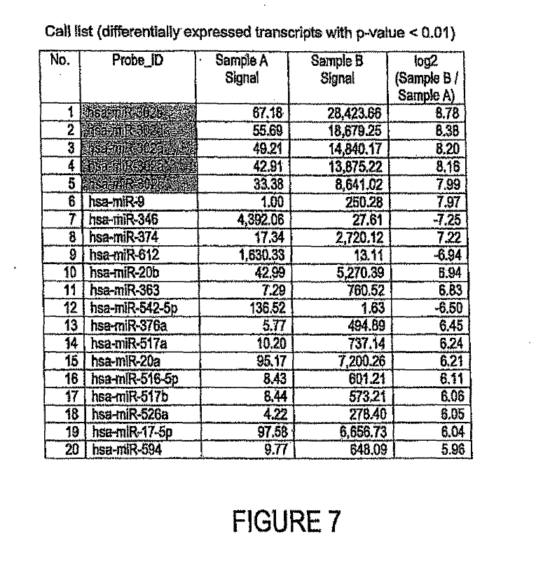

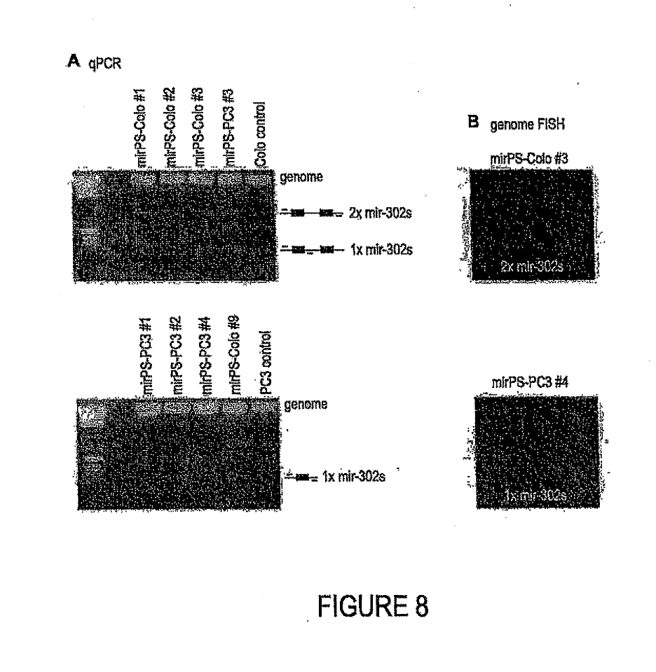

MirPS cells were able to form compact colonies reminiscent of embryoid bodies (EB) derived from human ES cells (FIG. 2F). When dissociated with collagenase IV and then cultivated in RPMI 1640 medium supplemented with 10% FBS, many of these EB-like cells differentiated into neuronal cells based on the presence of positive neuronal markers Tuj1 and ABCA2. We also noted that mirPS-PC3 EB cells could only differentiate into neuronal cell types, while mirPS-Colo EB cells formed teratoma-like primordial tissue structures in immunocompromised SCID-beige mice (FIG. 3), suggesting that different cancerous stem cells may have different pluripotent potentials. In view of the broad pluripotency in mirPS-Colo cells, we therefore evaluated the correlation between mir-302 and ES marker expression within the mirPS-Colo cells. As shown in FIG. 4A and FIG. 7, miRNA microarray analyses demonstrated that the expression rates of all mir-302s were significantly increased over eight folds in the mirPS-Colo cells. Since the four mir-302 members share very high homology and target almost the same cellular genes, this result indicates that the overall gene silencing effects of mir-302s may increase over thirty folds in the mirPS cells. Genomic PCR and fluorescent in situ hybridization assays further revealed that all mirPS cells carried either one or two copies of the mir-302s transgene (FIG. 8). Thus, the concentration of mir-302s may affect both pluripotency and survival of the mirPS cells.

### Identification of Human ES Cell Markers

Consistent with the elevation of mir-302s expression, many human ES cell markers were strongly detected in the mirPS cell, as determined by Western blot analyses (FIG. 4B). These ES markets, including Oct3/4, SSEA-3, SSEA-4, Sox2 and Nanog, were barely detected in both the original Colo cancer cells and the cells transfected with an empty pLNCX2-rT-SpRNAi vector (Colo+vector), a vector expressing mir-gfp miRNA (Colo+mir−gfp), or a vector expressing nonhomologous mir-434-5p pre-miRNA (Colo+mir-434-5p). Ben-Porath et al. (2008) have recently reported that many central regulators of ES identity, such as Oct3/4, Sox2 and Nanog, were not broadly expressed in high-grade cancers. Given that the concurrent expression of Oct3/4, SSEA-3 and SSEA-4 is a key determinant of human ES cell identity (Thomson et al., 1998), activation of these ES marker genes in mirPS-Colo cells indicates that mir-302s must provide certain “stemness” in order to reprogram the cancerous Colo cells into a human ES-cell-like state. As the miRBase::Sequences program at the website of microrna.sanger.ac.uk has predicted that methyl-CpG binding proteins, MECP2 and MECP1 component p66, are both strong targets of mir-302s, suppression of nuclear DNA methylation may be a mechanism underlying this reprogramming process.

### Assessment of Reprogramming-Related Genomic Demethylation

Change of epigenetic modification underlines another unique feature of ES cells, particularly genomic demethylation (Hochedlinger and Jaenisch, 2006). In order to reprogram a cell into its ES state, many embryonic genes need to be re-activated by DNA demethylation, such as Oct3/4. To assess this effect in the mirPS cells, we first performed a whole genome digestion with HpaII, a restriction enzyme that is sensitive to CpG methylation and cleaves only an unmethylated CCGG rather than methylated CCGG site. FIG. 4C shows that the digested DNA fragments from control Colo cells are over twice as large as those from the mirPS-Colo cells, indicating that the mirPS genome is highly demethylated. Further assessment in the Oct3/4 gene promoter region was performed using bisulfite PCR and genomic DNA sequencing (Takahashi and Yamanaka, 2006), which converted all unmethylated cytosines to uracils. Because unmethylated ACGT sites were also changed into AUGT sites by bisulfite, the digestion of mixed ACGT-cutting restriction enzymes failed to cleave this isolated region from the mirPS cells (FIG. 4D). The detailed demethylation maps shown by the bisulfite sequencing further demonstrated that over 90% methylation sties of the Oct3/4 gene promoter region were lost in the mirPS cells (FIG. 4E), suggesting that an epigenetic reprogramming process did occur to re-activate the Oct3/4 expression. Such epigenetic reprogramming is correlated to mir-302s expression because no DNA demethylation was found in cells transfected with an empty mir-302-free vector as compared to control cancer cells.

### Microarray Analysis of Genome-Wide Gene Expression Profiles

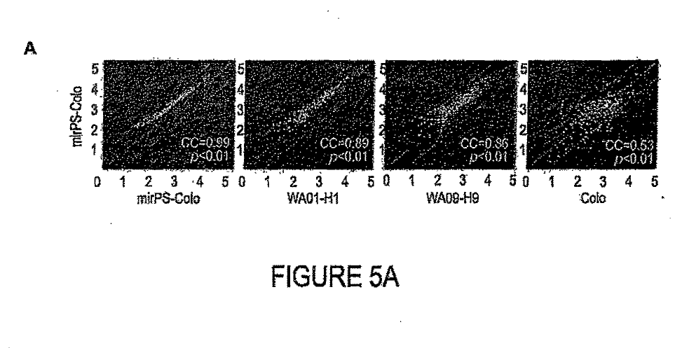

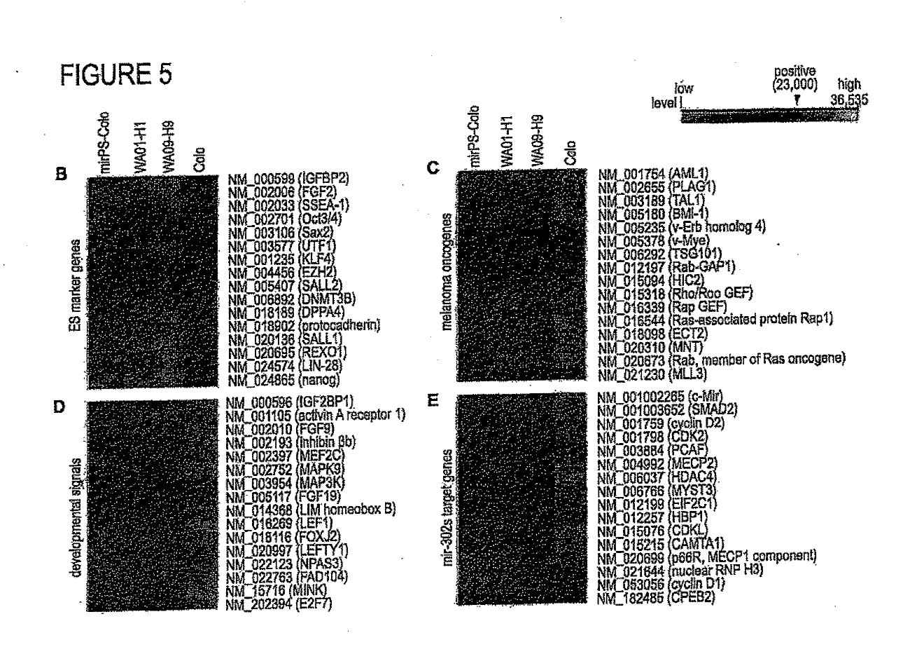

Genome-wide gene profiling was required to determine the genetic alterations associated with this mir-302-mediated reprogramming event. Microarray analysis was used to screen changes in genome-wide gene expression patterns in cells before and after the mir-302s transfection, as well as between the mirPS cells and human ES H1 and H9 cells. The changes in over 47,000 human gene expression patterns were assessed using Affymetrix gene microarrays (GeneChip U133A&B and U133 plus 2.0 arrays). We first duplicated the microarray tests using the same mirPS sample and selected two hundred most variable genes (white dots) from one of the tests for further comparison. As shown in FIG. 5A, the changes of these selected gene expressions were all less than one fold in the duplicated tests, indicating that the background variation was limited. Based on the scattering patterns of all microarray-identified genes, we then calculated the correlation coefficiency (CC) between the results of two compared transcriptome libraries. A CC rate was given to show the percentage of similarity in the genome-wide gene expression patterns with a threshold of only one-fold change. Under such stringent CC rate definition, we found that the gene expression patterns of mirPS-Colo cells were very similar to those of ES H1 (89%) and H9 (86%) cells, whereas only a low 53% CC rate was shown between Colo and mirPS-Colo cells. This strong genetic correlation between human ES and mirPS cells suggests that mir-302s may alter thousands of cellular gene expressions, which are involved in the reprogramming process of a cancer cell into an ES-like mirPS cell. For example, the elevation of many ES gene expressions and shutdown of numerous oncogenic, developmental and mir-302-targeted cell, cycle-related genes were consistently and concurrently observed in the mirPS and human ES results, as shown in FIGS. 5B-E.

In FIG. 5E, we noted that cell-cycle checkpoint genes, i.e., CDK2, cyclin D1 and D2, and DNA methylation facilitator, i.e., MECP2 and MECP1 component p66, were all confirmed to be strong targets for mir-302s. It is known that cyclin E-dependent CDK2 is required for the entry of S-phase cell cycle and inhibition of CDK2 results in G1-phase checkpoint arrest, whereas cyclin D1 can override G1-phase arrest in response to DNA damage (Sherr and Roberts, 2008). Based on this principle, the suppression of both CDK2 and cyclin D1 in mirPS cells revealed a fact that the cell cycle of mir-302s-transfected cancer cells could reach a very slow cell division rate as shown in FIG. 2D. The result of such cancer-stem cell cycle transition provides a significant benefit in cancer therapy. In addition, the suppression of MECP2 and MECP1/p66 activities was consistent with the results of FIGS. 4C-E, which indicated the epigenetic reprogramming of malignant cancer cells into benign mirPS cells. It is conceivable that the mirPS cells so obtained from patients may be further used to repair the cancer damages.

### In Vitro Molecular Guidance of mirPS Cell Differentiation

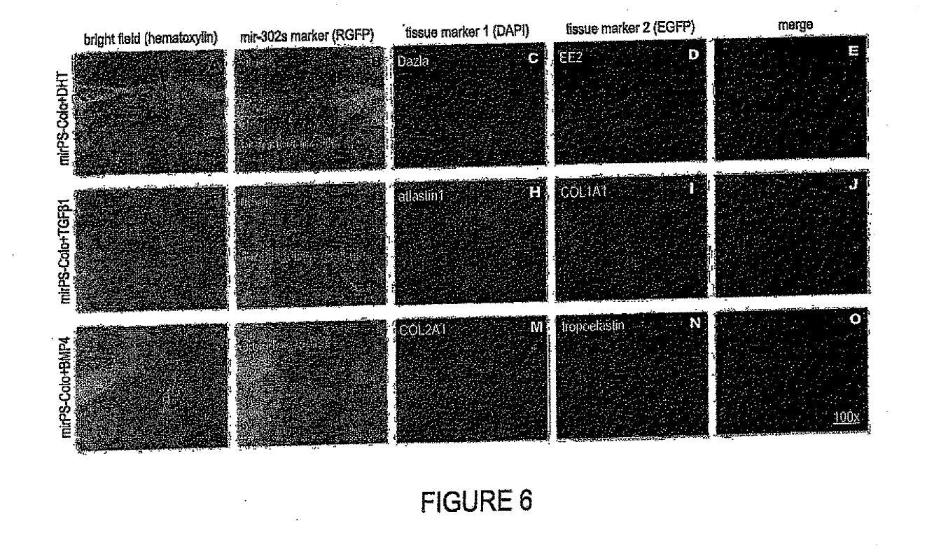

Pluripotency defines the most important characteristic of an ES cell. Through in vitro manipulations with different factors and/or hormones, human ES cells can differentiate into the three embryonic germ layers (ectoderm, mesoderm and definitive endoderm)—the founders of all adult tissues. In the absence of any treatment, xenograft implantation of the mirPS-Colo-derived embryoid bodies into the uterus or peritoneal cavity of female pseudopregnant immunocompromised SCID-beige mice formed teratoma-like primordial tissue structures (FIG. 8). The growth of these teratoma-like structures was terminated approximately 2.5-week post-implantation. It seems that there is a self-regulation mechanism limiting the random growth of these mirPS/EB cells in vivo. However, using in vitro treatments of various growth factors and/or hormones, we could direct the mirPS-Colo cells to differentiate into several tissue cell types ex vivo, including fibroblasts (FIGS. 6A-E), chondrocytes (FIGS. 6F-J) and spermatogonia-like (FIGS. 6K-O) primordial cells. The protocols for these in vitro conditions and xenotransplantation methods are provided in the Materials and Methods. Markers for the special tissue lineages were also identified with immunohistochemical detection, showing germ line-specific Dazla and EE2, fibroblast-specific atlastin1 and type I pro-collagen (COL1A1), and chondrocyte-specific tropoelastin and type II pro-collagen (COL2A1), respectively. These findings confirmed the pluripotency of the mirPS cells. It is conceivable that many more tissue cell types may be induced from these mirPS cells, using different molecular interventions.

### DISCUSSION

Ever since the first isolation of human ES cell line from human blastocysts (Thomson et al., 1998), there were concerns about destruction of human embryos, contamination of feeder cell antigens, and formation of teratomas. Recent reports on induced pluripotent stem (iPS) cells have opened up a new avenue for generating ES-like pluripotent cells directly from adult body cells, bypassing the use of human embryos as starting materials (Takahashi and Yamanaka, 2006; Takahashi et al., 2007). Using retroviral delivery of four transcription factor genes (i.e., either Oct4-Sox2-c-Myc-Klf4 or Oct4-Sox2-Nanog-Lin28) into mouse embryonic fibroblasts, the iPS cells so obtained were similar in many genetic and behavioral properties to mouse embryonic stem cells (Okita et al., 2007; Wernig et al., 2007). Additional iPS cell lines have continued to be developed from human embryonic fibroblasts and primary dermal fibroblast cultures using a similar approach (Yu et al., 2007; Park et al., 2008). Yet, there are two problems emerging from the process of iPS cell generation; one is the use of retroviral transgenes and the other the use of oncogenes (e.g., c-Myc and Klf4). Retroviral transfection is the only effective means to simultaneously and transgenically deliver the four full-length genes into a targeted somatic cell, whereas the random insertion of retroviral vectors into the transfected cell genome may also affect other non-targeted genes and produce unexpected results. This is problematic because simultaneous delivery of four large transgenes into one single cell is difficult to control, particularly when one or more of the genes are oncogenes.

Unlike the previous iPS cell technology, each member of the mir-302 family is able to simultaneously regulate over 445 cellular genes and they all share almost the same target genes based on the databases from the miRBase::Sequences program at the website of microrna.sanger.ac.uk. Many of the mir-302 targeted genes are active developmental signals involved in initiation or facilitation of lineage-specific, cell differentiation during early embryonic development. Thus, the function of mir-302s is more likely to attenuate the global production of developmental signals rather than to create transcriptional stimulation on certain embryonic signaling pathways. By inhibiting the cellular genes essential for embryonic development and cell differentiation, mir-302s is not only able to reprogram differentiated cancer cells into ES-like pluripotent stem cells but also to maintain their pluripotency and renewal under a feeder-free culture condition. Nevertheless, mir-302s may not be the only miRNA family involved in this mechanism because their target genes are also redundantly silenced by the group of mir-93, mir-367, mir-371, mir-372, mir-373 and mir-520 in human ES cells. Learning why these target genes must be simultaneously silenced during the reprogramming process of cancer-ES cell transformation may shed light on the mechanism underlying this miRNA-mediated gene silencing effect on ES cell maintenance and renewal.

Utilization of intronic mir-302 transfection provides a safe and powerful new tool for human ES-like pluripotent cell generation, particularly derived from cancerous and primarily cultured somatic cells. Because the intronic miRNA pathway is tightly regulated by multiple intracellular surveillance systems, such as mRNA transcription, RNA splicing, exosomal digestion and nonsense-mediated decay (NMD) mechanisms, it is considered to be much more effective, specific and safe than the siRNA/shRNA pathway (Lin et al., 2008). Advantageously, there are three breakthroughs in this mir-302-induced mirPS cell generation method. First, the transfection of a single mir-302s-expressing transgene offers a very simple, efficient and safe method for generating ES-like pluripotent stem cells, preventing the tedious retroviral insertion of all four large transcription factor genes into one single cell as demonstrated in the previous iPS methods. Second, because the size of the mir-302s-expressing transgene is just about 1 kilo-bases, the transfection efficiency is extremely high (almost 100%) and the selection of positive mirPS cells can be easily carried out by passing once through FACS flow cytometry, which is a very time-saving process. Third, the transfection process can be completed under a feeder-free condition without the risk of feeder antigen contamination and the mirPS cells so obtained can continue to grow in a feeder-free cultural condition. Fourth, no oncogene is used in this mirPS cell generation process. Lastly, we may use homologous DNA insertion in place of retroviral transfection to deliver the mir-302s-expressing transgene into a specific, desired region of the cell genome, preventing the risk of random insertion. Given that these advantages have solved most of the problems found in current stem cell research, these mirPS cells are useful for transplantation and cell therapies.

### MATERIALS AND METHODS

### Cell Culture and Treatments

Human cancer cell lines, Colo and PC3 cells were purchased from the American Type Culture Collection (ATCC, Rockville, Md.) and cultivated in RPMI 1640 medium supplemented with 10% fetal bovine serum (FBS), 4 mM L-glutamine, 1 mM sodium pyruvate, and 100 μg/ml gentamycin (Sigma Chemical, St. Louis, Mo.), at 37° C. under 5% CO₂. Cultures were passaged at ˜80% confluency by exposing cells to trypsin-EDTA solution for 1 min and rinsing once with RPMI, and the detached cells were replaced at 1:10 dilution in fresh growth medium. After mir-302 transfection, mirPS-Colo and mirPS-PC3 cells were grown on polyornithine/laminin-coated dishes, respectively, in a freshly made mirPS cell culture medium, containing either phenol red-free DMEM/F12 (1:1; high glucose) or RPMI 1640/B27 medium (Invitrogen, Carlsbad, Calif.) supplemented with 10% charcoal-stripped FBS, 4 mM L-glutamine, 1 mM sodium pyruvate, 5 ng/ml activin, 5 ng/ml noggin, 3 ng/ml bFGF (BD Biosciences, San Diego, Calif.) and an equal mixture of 0.5 μM Y-27632 and 0.5 μM GSK-3 inhibitor XV (EMD Biosciences, San Diego, Calif.), at 37° C. under 5% CO₂. The mirPS cells were passaged at 85%-90% confluency by exposing cells to trypsin-EDTA/collagenase IV (1 mg/ml) solution for 1 min and rinsing once with DMEM/F12 medium. Detached cells were replated at 1:3 dilution in fresh growth medium supplemented with 30% (v/v) conditioned medium which had exposed to the cells for 24 hour before passaging.

### Construction of the SpRNAi-RGFP Transgene Encoding an Intronic mir-302 Pre-miRNA Cluster Insert

The SpRNAi-RGFP transgene was generated as described below (Lin and Ying, 2006; Lin et, al., 2006). The intronic mir-302 pre-miRNA cluster consists of four parts: mir-302a, mir-302b, mir-302c and mir-302d pre-miRNAs. Synthetic oligonucleotides were: mir-302a-sense, 5′-GTCCGATCGT CCCACCACTT AAACGTGGAT GTACTTGCTT TGAAACTAAA GAAGTAAGTG CTTCCATGTT TTGGTGATGG ATCTCGAGCT C-3′; mir-302a-antisense, 5′-GAGCTCGAGA TCCATCACCA AAACATGGAA GCACTTACTT CTTTAGTTTC AAAGCAAGTA CATGCACGTT TAAGTGGTGG GACGATCGGA C-3′; mir-302b-sense, 5′-ATCTCGAGCT CGCTCCCTTC AACTTTAACA TGGAAGTGCT TTGTGTGACT TTGAAAGTAA GTGCTTCCAT GTTTTAGTAG GAGTCGCTAG CGCTA-3′; mir-302b-antisense, 5′-TAGCGCTAGC GACTCCTACT AAAACATGGA AGCACTTACT TTCAAAGTCA CAGAAAGCAC TTCCATGTTA AAGTTGAAGG GAGCGAGCTC GAGAT-3′; mir-302c-sense, 5′-CGCTAGCGCT ACCTTTGCTT TAACATGGAG GTACCTGCTG TGTGAAAGAG AAGTAAGTGC TTCCATGTTT CAGTGGAGGC GTCTAGACAT-3′; mir-302c-antisense, 5′-ATGTCTAGAC GCCTCCACTG AAACATGGAA GCACTTACTT CTGTTTCACA CAGCAGGTAC CTCCATGTTA AAGCAAAGGT AGCGCTAGCG-3′; mir-302d-sense, 5′-CGTCTAGACA TAACACTCAA ACATGGAAGC ACTTAGCTAA GCCAGGCTAA GTGCTTCCAT GTTTGAGTGT TCGACGCGTC AT-3′; and mir-302d-antisense, 5′-ATGACGCGTC GAACACTCAA ACATGGAAGC ACTTAGCCTG GCTTAGGTAA GTGCTTCCAT GTTTGAGTGT TATGTCTAGA CG-3′ (Sigma-Genosys, St. Louis, Mo.). We first hybridized mir-302a-sense to mir-302a-antisense, mir-302b-sense to mir-302b-antisense, mir-302c-sense to mir-302c-antisense, and mir-302d-sense to mir-302d-antisense, respectively, at 94° C. for 2 min, at 70° C. for 10 min and then at 4° C. in 1×PGR buffer. Then, the same amount of each hybrid of mir-302a, mir-302b, mir-302c, and mir-302d were digested with PvuI/XhoI, XhoI/NheI, NheI/XbaI, and restriction enzymes, respectively, at 37° C. for 4 hours. All of the digested hybrids were collected together with a gel extraction filter in 35 μl of autoclaved ddH₂O (Qiagen, Valencia, Calif.). Immediately after that, the mir-302 pre-miRNA cluster was formed by adding T4 DNA ligase (20 U) and buffer into the hybrid mixture and incubating the reaction at 12° C. for 12 hours. For intronic insertion, we mixed an equal amount (1:1) of the mir-302 pre-miRNA cluster and the SpRNAi-RGFP transgene, and then digested the mixture with PvuI and MluI restriction enzymes at 37° C. for 4 hours. The digested mixture was collected with a microcon-30 filter and ligated together with T4 DNA ligase (20 U) at 12° C. for 12 hours. This formed the SpRNAi-RGFP transgene with the intronic mir-302 pre-miRNA cluster insert.

### Incorporation of the SpRNAi-RGFP Transgene into a pLNCX2-rT-SpRNAi Retroviral Vector

We modified a VSV-G-positive pantropic retroviral vector, namely pLNCX2-rT, to transgenically deliver the mir-302-encoded SpRNAi-RGFP transgene (Lin et al., 2006), The pLNCX2-rT vector was derived from a modified pseudotype Moloney Murine Leukemia virus, pLNCX2 (Clontech, Palo Alto, Calif.). As shown in FIG. 1, we first incorporated the SpRNAi-RGFP transgene into the XhoI/AflII restriction site of the pLNCX2-rT vector and then inserted the mir-302 pre-miRNA cluster construct into the intronic insertion site. (PvuI/MluI restriction site) of the SpRNAi-RGFP transgene, so as to form a retroviral pLNCX2-rT-SpRNAi transgene vector capable of transgenically expressing mir-302s. In the experiments, we mixed an equal amount (1:1) of the SpRNAi-RGFP transgene and the pLNCX2-rT retroviral vector, and then digested the mixture with XhoI and AflII restriction enzymes at 37° C. for 4 hours. The digested mixture was collected with a microcon-30 filter and ligated together with T4 DNA ligase (20 U, Roche Biochemicals, Indianapolis, Ind.) at 12° C. for 12 hours. Following the same protocol except using PvuI/MluI digestion, we incorporated the mir-302 pre-miRNA cluster into the PvuI/MluI restriction site of the pLNCX2-rT-SpRNAi vector. The pre-miRNA-inserted pLNCX2-rT-SpRNAi vector was propagated E. coli DH5α LB cultures containing 100 μg/ml ampicillin (Sigma) and purified with a QIAprep spin miniprep kit (Qiagen), following the manufacturer's suggestion. For viral production, the pLNCX2-rT-SpRNAi vector was co-transfected with an equal amount of pVSV-G vector into GP2-293 packaging cells (Clontech) to produce infectious, but not replicable, pantropic retroviruses. GP2-293 cells were grown in phenol red-free DMEM medium supplemented with 10% FBS, 4 mM L-glutamine and 1 mM sodium pyruvate. The cotransfection was carried out with a FuGene 6 reagent (Roche), following the manufacturer's suggestion.

### MirPS Cell Generation Using the pLNCX2-rT-SpRNAi Transgene Vector

High titer viruses were released in the DMEM medium of the GP2-293 cell cultures approximately 36-48 hours after the co-transfection of pVSV-G and pLNCX2-rT-SpRNAi vectors. The viral titer was measured to be over multiplicity of infection (MOI) 30 before call transfection, following the protocol of a retro-X qRT-PCR titration kit (Clontech). Then, we transferred the high titer virus medium to the Colo or PC3 cell cultures and incubated the cells for 12 hours at 37° C. under 5% CO₂. After that, fresh mirPS cell culture medium was added in place of the virus medium and replaced every three days. Positively transfected cells were isolated and collected 24 hours post-infection, using FACS flow cytometry sorting with a monoclonal antibody against the mir-302 expression maker. RGFP (Clontech). These isolated sells were grown in the mirPS cell culture medium as aforementioned.

### In Vitro Molecular Guidance of the mirPS Cell Differentiation

In absence of any treatment except the feeder-free mirPS culture medium, xenograft implantation of the mirPS cells into the uterus or peritoneal cavity, but not other tissues, of female pseudopregnant immunocompromised SCID-beige mice could form teratoma-like primordial tissues. The use of immunocompromised nude mice was to provide an in vivo environment mimicking transplantation therapy. The pseudopregnant mice were made by intraperitoneally injection of 1 IU human menopausal gonadotrophin (HMG) for two days and then human chorionic gonadotrophin (hCG) for one more day. For in vitro molecular guidance into spermatogonia lineage, mirPS cells were maintained on polyornithine/laminin-coated dishes in DMEM/F12 (1:1; high glucose) medium supplemented with charcoal-stripped 10% FBS, 4 mM L-glutamine, 1 mM sodium pyruvate, 5 ng/ml activin and 50 ng/ml dihydrotestosterone (DHT) for 12 hours, at 37° C. under 5% CO₂. Then the cells were trypsinized, washed with 1×PBS, and collected in four aliquots of chilled Matrigel (100 μl each) and one aliquots of 100 μl 1×PBS. Immediately after that, we transplanted the cells into the hind limb muscle, peritoneum, uterus, subcutaneous neck skin (with Matrigel) and tail vein (with PBS) of 6-week-old athymic immunocompromised SCID-beige nude mice. The mice were anesthetized with diethyl ether during experimental processing. One week later, spermatogonia-like cells were found only in the uterus area. For fibroblast/differentiation, we followed the same procedure as shown above, except using regular phenol red-free DMEM medium supplemented with 10% FBS, 4 mM L-glutamine; 1 mM sodium pyruvate, 5 ng/ml noggin and 100 ng/ml transforming growth factor beta1 (TGF-β1) for 6 hours before xenotransplantation. Fibroblast-like cells were found in the uterus one week later. For chondrocyte differentiation, we performed the same procedure as before but using regular RPMI 1640 medium supplemented with 10% FBS, 4 mM L-glutamine, 1 mM sodium pyruvate and 100 ng/ml bone morphogenetic protein 4 (BMP4) for 6 hours. Chondrocyte-like cells were found only in the liver area.

### REFERENCES

Bartel, D. P. 2004. MicroRNAs: genomics, biogenesis, mechanism, and function. Cell 116:281-287.

Ben-Porath, I., Thomson, M. W., Carey, V. J., Ge, R., Bell, G. W., Regev, A., and Weinberg R. A. 2008. An embryonic stem cell-like gene expression signature in poorly differentiated aggressive human tumors. Nat. Genet. 40:499-507.

Danin-Kreiselman, M., Lee, C. Y., and Chanfreau, G. 2003. RNAse III-mediated degradation of unspliced pre-mRNAs and lariat introns. Mol. Cell 11:1279-1289.

Hochedlinger, K., and Jaenisch, R. 2006. Nuclear reprogramming and pluripotency. Nature 441:1061-1067.

Lin, S. L., Chang, D., Wu, D. Y., and Ying, S. Y. 2003. A novel RNA splicing-mediated gene silencing mechanism potential for genome evolution. Biochem Biophys Res Commun. 310:754-760.

Lin, S. L., and Ying, S. Y. 2006. Gene silencing in vitro and in vivo using intronic microRNAs. Shao-Yao Ying (Ed.) MicroRNA protocols, Humana press, Totowa, N.J., pp295-312.

Lin, S. L., Chang, S. J. E., and Ying, S. Y. 2006. Transgene-like animal model using intronic microRNAs. Methods Mol Biol. 342:321-334.

Lin, S. L., Kim, H., and Ying, S. Y. 2008. Intron-mediated RNA interference and microRNA (miRNA). Front. Biosci. 13:2216-2230.

Okita, K., Ichisaka, T., and Yamanaka, S. 2007. Generation of germline-competent induced pluripotent stem cells. Nature 448:313-317.

Park, L. H., Zhao, R., West, J. A., Yabuuchi, A., Huo, H., Ince, T. A., Lerou, P. H., Lensch, M. W., and Daley, G. Q. 2008. Reprogramming of human somatic cells to pluripotency with defined factors. Nature 451:141-146.

Reya, T., Morrison, S. J., Clarke, M. F., and Weissman, I. L. 2001. Stem cells, cancer, and cancer stem cells. Nature 414:105-111.

Rodriguez, A., Griffiths-Jones, S., Ashurst, J. L., and Bradley, A. 2004. Identification of mammalian microRNA host genes and transcription unite. Genome Res. 14:1902-1910.

Ruby, J. G., Jan, C. H., and Bartel, D. P. 2007. Intronic microRNA precursors that bypass Drosha processing. Nature 448:83-86.

Sherr, C. J., and Roberts, J. M., 2008. Living with or without cyclins and cyclin-dependent kinases. Genes Dev. 18:2699-2711.

Suh, M. R., Lee, Y., Kim, J. Y., Kim, S. K., Moon, S. H., Lee, J. Y., Cha, K. Y., Chung, H. M., Yoon, H. S., Moon, S. Y., Kim, V. N., and Kim, K. S. 2004. Human embryonic stem cells express a unique set of microRNAs. Dev. Biol. 270:488-498.

Takahashi, K., and Yamanaka, S. 2006. Induction of pluripotent stem cells from mouse embryonic and adult fibroblast cultures by defined factors. Cell 126:663-676.

Takahashi, K., Tanabe, K., Ohnuki, M., Narita, M., Ichisaka, T., Tomoda, K., and Yamanaka, S. 2007. Induction of pluripotent stem cells from adult human fibroblasts by defined factors. Cell 131:861-872.

Thomson, J. A., Itskovitz-Eldor, J., Shapiro; S. S., Waknitz, M. A., Swiergiel J. J., Marshall, V. S., and Jones, J. M. 1998. Embryonic stem cell lines derived from human blastocysts. Science 282: 1145-1147.

Wernig, M., Meissner, A., Foreman, R., Brambrink, T., Ku, M., Hochedlinger, K., Bernstein, B. E., and Jaenisch, R. 2007 In vitro reprogramming of fibroblasts into a pluripotent ES-cell-like state. Nature 448: 318-324.

Xia, X. G., Zhou, H., Samper, E., Melov, S., and Xu, Z. 2006. Pol II-expressed shRNA knocks down Sod2 gene expression and causes phenotypes of the gene knockout in mice. PLoS Genet. 2: e10.

Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., Slukvin, I. L., and Thomson, J. A. 2007. Induced pluripotent stem cell lines derived from human somatic cells. Science 318: 1917-1920.

### EXAMPLE II

### Materials and Methods

Construction of the SpRNAi-RGFP transgene. The SpRNAi-RGFP transgene was generated as reported (1, 2, 3), consisting of three parts: one artificial intron, namely SpRNAi, and two exons derived from a mutated red fluorescent HcRed1 chromoprotein gene isolated from Heteractis crispa, namely RGFP. Synthetic oligonucleotides used for generating the SpRNAi intron were: sense phosphorylated 5′-GTAAGTGGTC CGATCGTCGC GACGCGTGAT TACTAACTAT CAATATCTTA ATCCTGTCCC TTTTTTTTCC ACAGTAGGAC CTTCGTGCA-3′ and antisense 5′-TGCACGAAGG TCCTACTGTG GAAAAAAAAG GGACAGGATT AAGATATTGA TAGTTAGTAA TGACGCGTCG CGACGATCGG ACCACTTAC-3′ (Sigma-Genosys, St. Louis, Mo.). The SpRNAi intron was formed by hybridization of an equal mixture (1:1) of each sequence at 94° C. for 2 min, at 70° C. for 10 min and then at 4° C. in 1×PCR buffer (e.g., 50 mM Tris-HCl, pH 9.2 at 25° C., 16 mM (NH₄)₂SO₄, 1.75 mM MgCl₂). The hybridized SpRNAi intron was purified with a microcon-30 filter (Amicon, Beverly, Mass.) in 10 μl of autoclaved ddH₂O, and than digested with a DraII restriction enzyme (10 U) at 37° C. for 4 hours. The digested intron was collected with a new microcon-30 filter in 10 μl of autoclaved ddH₂O. Concurrently, two RGFP exon sequences were generated by enzymatic cleavage with DraII in the 208th nucleotide (nt) site of the HcRed1 gene (BD Biosciences, Palo Alto, Calif.) and the 5′-end exon fragment was further blunt-ended by T4 DNA polymerase (5 U). After that, the SpRNAi-RGFP transgene was formed by ligation of the SpRNAi intron and the two RGFP exons. We first mixed an equal mixture (1:1:1) of the intron and exons and incubated the mixture in 1×PCR buffer from 50° C. to 10° C. over a period of 1 hour. Then T4 DNA ligase (20 U) and buffer (Roche Biochemicals, Indianapolis, Ind.) were added into the mixture and the ligation was carried out at 12° C. for 12 hours. For cloning the full-length SpRNAi-RGFP transgene, the ligated products (10 ng) were amplified by high-fidelity PCR (Roche) with primers (sense 5′-CTCGAGCATG GTGAGCGGCC TGCTGAA-3′ and antisense 5′-dTCTAGAAGTT GGCCTTCTCG GGCAGGT-3′) at 94° C. for 1 min, at 54° for 1 min and then at 68° C. for 2 min. for 25 cycles. The resulting PCR products were fractionated on a 2% agarose gel and a ˜900 base-pair (bp) sequence was extracted and purified by a gel extraction kit (Qiagen, Valencia, Calif.), following the manufacturer's suggestion. The nucleotide composition of the SpRNAi-RGFP transgene was confirmed by DNA sequencing.

Flow Cytometry assay. Cells were trypsinized, pelleted and fixed by re-suspending in 1 ml of pre-chilled 70% methanol in PBS for 1 hour at −20° C. The cells were pelleted and washed once with 1 ml of PBS. The cells were pelleted again and resuspended in 1 ml of 1 mg/ml propidium iodide, 0.5 mg/ml RNase in PBS for 30 min at 37° C. Approximately 15,000 cells were then analyzed on a BD FACSCalibur flow cytometer (San Jose, Calif.). Cell doublets were excluded by plotting pulse width versus pulse area and gating on the single cells. The collected data were analyzed using the software package Flowjo using the “Watson Pragmatic” algorithm (4). The first (left) and second (right) peaks of the flow cytometry charts represented the levels of resting G0/G1 and mitotic M phase cell populations in the entire tested cell population, respectively.

Immunodetection assay. Embedding and sectioning tissue samples were performed as previously reported (1, 2). Briefly, the samples were fixed in 4% paraformaldehyde overnight at 4° C. The samples were washed sequentially with 1×PBS, methanol, isopropanol and tetrahydronaphthalene before embedded in paraffin wax. The embedded samples were then cut on a microtome at 7-10 μm thickness and mounted on clean TESPA-coated slides. Then, the slides were dewaxed with xylene and mounted under coverslips using mounting media (Richard Allan Scientific, Kalamazoo, Mich.) and stained by hematoxylin and eosin (H&E, Sigma) for morphological observation. Immunohistochemical (IHC) staining was performed as reported (1). Immunohistochemical staining kits were purchased from Imgenex (San Diego, Calif.). Processes for antibody dilution and immunostaining were performed according to the manufacturers' suggestions. Primary antibodies used included Tuj1 (1:500, Abcam Inc., Cambridge, Mass.), ABCA2 (1:100, Santa Cruz Biotechnology, Santa Cruz, Calif.), Dazla (1:100, Abcam), EE2 (1:100, Santa Cruz), atlastin1 (1:200, Santa Cruz), COL1A1 (1:500, Santa Cruz), COL2A1 (1:500, Santa Cruz), tropoelastin (1:200, Abcam), and RGFP (1:500, Clontech). Fluorescent dye-labeled goat anti-rabbit or horse anti-mouse antibody was used as the secondary antibody (1:2,000, Invitrogen-Molecular Probes). Positive results were observed under a 100× microscope with whole field scanning and measured at 200× or 400× magnification for quantitative analysis by a Metamorph Imaging program (Nikon 80i and TE2000 microscopic quantitation systems).

Western blot analysis. Western blotting of protein targets was performed as previously reported (1). Cells at ˜70% confluency were lysed with a CelLytic-M lysis/extraction reagent (Sigma) supplemented with protease inhibitors, Leupeptin, TLCK, TAME and PMSF, following the manufacturer's suggestion. The total protein volume was determined using an improved SOFTmax protein assay package on an E-max microplate Header (Molecular Devices, CA). Each 30 μg of cell lysate was added to SDS-PAGE sample buffer under reducing (+50 mM DTT) and non-reducing (no DTT) conditions, and boiled for 3 min before loading onto 6˜8%. polyacylamide gels; molecular weights were determined by comparison to standard proteins (Bio-Rad, Hercules, Calif.). SDS-polyacrylamide gel electrophoresis was performed according to the standard protocols (5). Proteins resolved by PAGE were electroblotted onto a nitrocellulose membrane and incubated in Odyssey blocking reagent (Li-Cor Biosciences, Lincoln, NB) for 2 hours at room temperature. Then, we applied a primary antibody to the reagent and incubated the mixture at 4° C. Primary antibodies used included Oct3/4 (1:500, Santa Cruz), SSEA-3 (1:500, Santa Cruz), SSEA-4 (1:500, Santa Cruz), Sox2 (1:500, Santa Cruz), Nanog (1:500, Santa Cruz), Klf4 (1:200, Santa Cruz), β-actin (1:2000, Chemicon, Temecula, Calif.), and RGFP (1:1000, Clontech). After overnight, the membrane was rinsed three times with TBS-T and then exposed to goat anti-mouse IgG conjugated secondary antibody to Alexa Fluor 680 reactive dye (1:2,000; Invitrogen-Molecular Probes), for 1 hour at the room temperature. After three additional TBS-T rinses, fluorescent scanning of the immunoblot and image analysis were conducted using Li-Cor Odyssey Infrared Imager and Odyssey Software v.10 (Li-Cor).

Fluorescent in situ hybridization (FISH). The FISH assay kit was purchased from Ambion Inc. (Austin, Tex.) and performed according to the manufacturer's suggestions. We used a synthetic locked nucleic acid [LNA]-DNA probes (Sigma-Genosys) directed against the junction region between RGFP 5′-exon and mir-302-inserted intron (5′-CCTGGCCCCC TGCTGCGAGT ACGGCAGCAG GACGTAAGTG GATCCGATCG TCCCACCACT TAAACGTGGA TGTACTTGCT TTGAAACTAA A-3′). Cells were cultivated on polyornithine/laminin-coated cultural slides. At 30%-40% confluency, cells were pre-fixed in 4% paraformaldehyde for 30 min, then digested with proteinase K and RNase A (10 μg/mL Roche) for 10 min at 37° C., re-fixed with 4% paraformaldehyde, and washed in Tris/glycine buffer. Nuclear membranes were dissolved with a detergent buffer (10 mM Tris-HCl, pH 7.4, 100 mM NaCl, 5 mM MgCl₂, 1 mM EDTA, 4 mM vanadyl adenosine, 1.2 mM phenylmethylsulfonyl fluoride, 1% (v/v) Tween 40, and 0.5% (v/v) sodium deoxycholate) for 5 min at 4° C. and washed three times in Tris/glycine buffer. After that, the slides were hybridized overnight at 60° C. within cloverslip chambers in in situ hybridization buffer (40% formamide, 5× SSC, 1× Denhard's solution. 100 μg/ml salmon testis DNA, 100 μg/ml tRNA), containing 1 ng/μl of Alexa Fluro 647-labeled LNA-DNA probes. After post-hybridization washes once with 5× SSC and once with 0.5× SSC at 25° C. for 1 hour, positive results were observed under a 100× microscope with whole field scanning and recorded at 200× and 1,000× magnification (Nikon 80i microscopic quantitation system).

Bisulfite PCR and genomic DNA sequencing. Genomic DNAs from about two million cells were isolated with a DNA isolation kit (Roche) and divided into two aliquots. One of the DNA aliquot (2 μg) was digested with a CCGG-cutting restriction enzyme, HpaII, and then assessed with 1% agarose gel electrophoresis to determine genome-wide demethylation. The other aliquot (2 μg) was used for PCR cloning the complete 9,400 base-pair (bp) 5′-regulatory region of the Oct3/4 promoter (NT_{—}007592 nucleotides 21992184-22001688), before and after bisulfite modification. Bisulfite modification was performed with a CpGenome DNA modification kit (Chemicon), according to the manufacturers' suggestions. The treatment of bisulfite to DNA converted all unmethylated cytosines to uracils while methylated cytosines remained as cytosines. For example, unmethylated ACGT sites, but not methylated ACGT, were changed into AUGT sites. PCR primers specific to the target Oct3/4 5′-promoter region before and after bisulfite modification had been designed and tested in the Takahashi's report (6), including two forward primers 5′-GAGGAGTTGA GGGTAGTGTG-3′ (for bisulfite-modified DNAs) and 5′-GAGGAGCTGA GGGCACTGTG-3′ (for non-modified DNAs) and one reverse primer 5′-GTAGAAGTGC CTCTGCCTTC C-3′. For PCR cloning, the genomic DNAs (50 ng), either bisulfite-treated or untreated, were first mixed with the primers (total 150 pmole) in 1× PCR buffer, heated to 94° C. for 4 min, and immediately cooled on ice. After that, 25 cycles of PCR were performed as follows: at 92° C. for 1 min, at 55° C. for 1 min and then at 70° C. for 5 min, using a long template PCR extension kit (Roche). The resulting products were collected with a PCR purification kit (Qiagen) and 2 μg of the DNAs were digested with an equal mixture (5 U each) of multiple ACGT-cutting restriction enzymes, containing AclI (AACGTT), BmgBI (CACGTC), PmlI (CACGTG), SnaBI (TACGTA) and HpyCH4IV (ACGT). Then the digested fragments were assessed using 3% agarose gel electrophoresis. For DNA sequencing analysis, we further amplified a 467-bp target region flanking the Oct3/4 transcription initiation site (NT_{—}007592 nucleotides 21996577-21997043), using quantitative PCR (qPCR). Primers used were one forward primer 5′-GAGGCTGGAG TAGAAGGATT GCTTTGG-3′ and one reverse primer 5′-CCCTCCTGAC CGATCACCTC CACCACC-3′. The above PCR-cloned Oct3/4 5′promoter region (50 ng) were mixed with the primers (total 100 pmole) in 1× PCR buffer, heated to 94° C. for 2 min, and immediately cooled on ice. Then, 20 cycles of PCR were performed as follows: at 94° C. for 30 sec and at 68° C. for 1 min, using a high-fidelity PCR extension kit (Roche). The amplified DNA products with a correct 467-bp size were further fractionized by 3% agarose gel eleotrophoresis, purified with a gel extraction kit (Qiagen), and then used in DNA sequencing. A detailed profile of the DNA methylation sites was generated by comparing the unchanged cytosines in the bisulfite-modified DNA to those in the non-modified DNA sequence.

MicroRNA microarray analysis (p<0.01, n=3). At 70% confluency, small RNAs from each cell culture were isolated, using the mirVANA™ miRNA isolation kit (Ambion) following the manufacturer's suggestion. The purity and quantity of the isolated small RNAs were assessed, using 1% formaldehyde-agarose gel electrophoresis, and spectrophotometer measurement (Bio-Rad), and then immediately frozen in dry ice and submitted to LC Sciences (San Diego, Calif.) for miRNA microarray analysis. Each microarray chip was hybridized with a single sample labeled with either Cy3 or Cy5 or a pair of samples labeled with Cy3 and Cy5, respectively. Background subtraction and normalization were performed. For a dual sample assay, a p-value calculation was performed and a list of differentially expressed transcripts more than 3-fold was produced. In the Cy3 and Cy5 intensity images (blue background), as signal intensity increased from level 1 to level 65,535 the corresponding color changed from blue to green, to yellow, and to red. The levels above 23,000 were considered to be positive calls in gene expression. In the Cy5/Cy3 ratio image (black background), when Cy3 level was higher than Cy5 level the color was green; when Cy3 level was equal to Cy5 level the color was yellow; and when Cy5 level was higher than Cy3 level the color was red.

Genome microarray analysts (p<0.01, n=4). Human genome GeneChip U133A&B and plus 2.0 arrays (Affymetrix, Santa Clara, Calif.) containing over 54,000 oligonucleotide probes were used to detect the expression patterns of genome-wide 47,000 human gene transcripts in mirPS cells. Total RNAs from each tested sample were isolated using RNeasy spin columns (Qiagen). To prepare labeled probes for microarray hybridization, the extracted total RNAs (2 μg) were converted into double-stranded cDNAs with a synthetic oligo(dT)₂₄-T7 promoter primer, 5′-GGCCAGTGAA TTGTAATACG ACTCACTATA GGGAGGGGG-(dT)₂₄-3′, using Superscript Choice system (Invitrogen). The resulting cDNAs were purified by phenol/chloroform extractions, precipitated with ethanol, and resuspended at a concentration of 0.5 μg/μl in diethyl pyrocarbonate (DEPC)-treated ddH₂O. Then, in vitro transcription was performed, containing 1 μg of the dsDNAs, 7.5 mM unlabeled ATP and GTP, 5 mM unlabeled UTP and CTP, and 2 mM biotin-labeled CTP and UTP (biotin-11-CTP, biotin-16-UTP, Enzo Diagnostics), and 20 U of T7 RNA polymerase. Reactions were carried out for 4 hours at 37° C. and the resulting cRNAs were purified by RNeasy spin columns (Qiagen). A part of the cRNA sample was separated on a 1% agarose gel to check the size range, and then 10 μg of the cRNAs were fragmented randomly to an average size of 50 bases by heating at 94° C. for 35 min in 40 mM Tris-acetate, pH 8.0, 100 mM KOAc/30 mM MgOAc. Hybridizations were completed in 200 μl of AFFY buffer (Affymetrix) at 40° C. for 16 hours with constant mixing. After hybridization, arrays were rinsed three times with 200 μl of 6× SSPE-T buffer (1× 0.25 M sodium chloride/15 mM sodium phosphate, pH 7.6/1 mM EDTA/0.005% Triton) and then washed with 200 μl of 6× SSPE-T for 1 hour at 50° C. The arrays were further rinsed twice with 0.5× SSPE-T and washed with 0.5× SSPE-T at 50° C. for 15 min. Then, staining assays were done with 2 μg/ml streptavidinphycoerythrin (Invitrogen-Molecular Probes) and 1 mg/ml acetylated BSA (Sigma) in 6× SSPET (pH 7.6). The arrays were read at 7.5 μm with a confocal scanner (Molecular Dynamics). To identify the background variations, we duplicated the microarray tests using the same sample and selected two hundred genes, which were slightly presented in one side of the tests, for further comparison. The samples were normalized using the total average difference between perfectly matched probes and mismatched probes. Then, alterations of overall genome-wide gene expression patterns were analyzed using Affymetrix Microarray Suite version 5.0, Expression Console™ version 1.1.1 (Affymetrix) and Genesprings (Silicon Genetics) softwares. Changes in gene expression rates more than 1-fold were considered as positive differential genes. In gene clustering assays, as signal intensity increased from level 1 to level 65,535 the corresponding color changed from green to black, and to red. The level above 23,000 (in red) was considered to be a positive call in individual gene expression.

After retroviral infection (˜12 hours), the medium is changed to the mirPS cell medium as reported (Lin et al., 2008, RNA 14:2115-2124). Noggin may not be needed for Borne somatic cell types. Three days later, positively infected cells are selected using FACS with an antibody against RGFP (or any marker used for co-expression with the mir-302 cluster). It takes about one or two more weeks to see small colonies. If the mir-302 cluster is successfully expressed in the infected cells, 95%-98% apoptotic tumor cells will be seen due to the strong silencing of CDK2 and cyclin D1 and D2 (over 80% reduction) by mir-302. Such strong apoptotic effect will not occur in mirPS cells derived from normal somatic cells. As shown in Lin et al., 2008, RNA 14:2115-2124, CDK2 and cyclin D1 and D2, are valid targets for mir-302. Therefore, only a small percentage of the cells survive after the infection.

If outgrowth of prostate cancer PC3 or melanoma Colo-829 cells is observed in the cultures, it is very likely that either the viral titer used is too low or the mir-302 is not properly expressed, or both. The retroviral titer used is preferably over MOI 30. The higher, the better; however, if it is too high, all the cells will be arrested at the G1 phase. Thus, the concentration should be optimized for the cell condition. Preferably, the Pol III- or CMV-driven siRNA/shRNA direct expression systems are not employed due to their low expression rates in Colo/PC3 cells (˜16 fold increase in total). This is probably due to the short template and highly structured conformation of the mir-302 cluster (˜350 bp) which may be difficult to be, directly transcribed. Preferably, mir-302 is not expressed with an eGFP marker, which exhibits certain toxicity at a high concentration.

After colonies are observed (one to two weeks after FACS selection), each colony should be isolated into a different 98-well plate for continued growth for up to one month. The colonies will form blastula-like embryoid bodies (EB). Up to this stage, they can be either used for assays or dissociated for sub-culturing. See the Figures for mirPS-Colo cell growth (similar to mirPC3). Early-stage EB should be used for the experiments. The mirPS cells after the mature-stage EB may contain some apoptotic cells in the center of the EB.

### REFERENCES

1 Lin, S. L., Chang, D., Wu, D. Y., and Ying, S. Y. 2003. A novel RNA splicing-mediated gene silencing mechanism potential for genome evolution. Biochem. Biophys. Res. Commun. 310: 754-760.

2 Lin, S. L., and Ying, S. Y. 2006. Gene silencing in vitro and in vivo using intronic microRNAs. Methods Mol Biol. 342: 295-312.

3 Lin, S. L., Chang, S. J. E., and Ying, S. Y. 2006. Transgene-like animal model using intronic microRNAs. Ying, S. Y. (Ed.) MicroRNA protocols, pp. 321-343, Humana press, Totowa, N.J.

4 Watson, J. V., Chambers, S. H., and Smith, P. J. 1987. A pragmatic approach to the analysis of DNA histograms with a definable G1 peak. Cytometry 8: 1-8.

5 Sambrook, J., and Russell, D. W. 2001. Molecular Cloning, 3 Ed. Cold Spring Harbor Laboratory press, Cold Spring Harbor, N.Y.

6 Takahashi, K., Tanabe, K., Ohnuki, M., Narita, M., Ichisaka, T., Tomoda, K., and Yamanaka, S. 2007. Induction of pluripotent stem cells from adult human fibroblasts by defined factors. Cell 131: 861-872.

All publications cited herein are incorporated by reference in their entirety.

What is claimed is:

1. An isolated nucleic acid comprising one or more mir (microRNA)-302 genes operably linked to a regulatory sequence, wherein the regulatory sequence controls the expression of the mir-302 genes.

2. The nucleic acid of claim 1, wherein the mir-302 genes are selected from the group consisting of the mir-302a, mir-302b, mir-302c, and mir-302d gene.

3. An isolated nucleic acid comprising a regulatory sequence operably linked to a recombinant sequence encoding a contiguous transcript,
wherein the regulatory sequence controls the transcription of the recombinant sequence,
wherein the recombinant sequence comprises a first gene encoding at least two exons flanking one intron,
wherein the intron comprises one or more mir-302s, and
wherein the intron is spliced out of the contiguous transcript of the recombinant sequence to allow the mir-302s to interact with their targets in a cell.

4. The nucleic acid Of claim 3, wherein the mir-302s are selected from the group consisting of mir-302a, mir-302b, mir-302c, and mir-302d.

5. The nucleic acid of claim 3, wherein the nucleic acid is transcribed by a type II RNA polymerase.

6. The nucleic acid of claim 3, wherein the intron is spliced out of the contiguous transcript of the recombinant sequence by a spliceosome.

7. The nucleic acid of claim 3, wherein the first gene is the RGFP (red fluorescent HcRed1 chromoprotein) gene or a fragment thereof.

8. An expression vector comprising the nucleic acid of claim 1 or 3.

9. A host cell comprising the nucleic acid of claim 1 or 3.

10. A transgenic animal comprising the nucleic acid of claim 1 or 3.

11. A method of generating ES (embryonic stem)-like cells, comprising contacting non-ES-like cells with the nucleic acid of claim 1 or 3, thereby transforming the non-ES-like cells into ES-like cells.

12. The method of claim 11, wherein the non-ES-like cells are cancer cells.

13. The method of claim 12, wherein the non-ES-like cells are Colo or PC3 cells.

14. The method of claim 11, further comprising inducing the ES-like cells to differentiate into tissue cell types.

15. The method of claim 14, wherein the non-ES-like cells are cancer cells.

16. The method of claim 15, wherein the non-ES-like cells are Colo or PC3 cells.

17. The method of claim 16, wherein the ES-like cells form a teratoma-like primordial tissue structure, fibroblasts, chondrocytes, spermatogonia-like primordial cells, or neuronal cells.


SEQUENCE LISTING (20 sequences)

SEQ ID NO: 1 - DNA, 91 bp, Artificial Sequence
  Synthetic oligonucleotide
gtccgatcgtcccaccacttaaacgtggatgtacttgctttgaaactaaagaagtaagtgcttccatgttttggtgatggatctcgagctc

SEQ ID NO: 2 - DNA, 91 bp, Artificial Sequence
  Synthetic oligonucleotide
gagctcgagatccatcaccaaaacatggaagcacttacttctttagtttcaaagcaagtacatccacgtttaagtggtgggacgatcggac

SEQ ID NO: 3 - DNA, 95 bp, Artificial Sequence
  Synthetic oligonucleotide
atctcgagctcgctcccttcaactttaacatggaagtgctttctgtgactttgaaagtaagtgcttccatgttttagtaggagtcgctagcgcta

SEQ ID NO: 4 - DNA, 95 bp, Artificial Sequence
  Synthetic oligonucleotide
tagcgctagcgactcctactaaaacatggaagcacttactttcaaagtcacagaaagcacttccatgttaaagttgaagggagcgagctcgagat

SEQ ID NO: 5 - DNA, 90 bp, Artificial Sequence
  Synthetic oligonucleotide
cgctagcgctacctttgctttaacatggaggtacctgctgtgtgaaacagaagtaagtgcttccatgtttcagtggaggcgtctagacat

SEQ ID NO: 6 - DNA, 90 bp, Artificial Sequence
  Synthetic oligonucleotide
atgtctagacgcctccactgaaacatggaagcacttacttctgtttcacacagcaggtacctccatgttaaagcaaaggtagcgctagcg

SEQ ID NO: 7 - DNA, 82 bp, Artificial Sequence
  Synthetic oligonucleotide
cgtctagacataacactcaaacatggaagcacttagctaagccaggctaagtgcttccatgtttgagtgttcgacgcgtcat

SEQ ID NO: 8 - DNA, 82 bp, Artificial Sequence
  Synthetic oligonucleotide
atgacgcgtcgaacactcaaacatggaagcacttagcctggcttagctaagtgcttccatgtttgagtgttatgtctagacg

SEQ ID NO: 9 - DNA, 89 bp, Artificial Sequence
  Synthetic oligonucleotide
gtaagtggtccgatcgtcgcgacgcgtcattactaactatcaatatcttaatcctgtcccttttttttccacagtaggaccttcgtgca

SEQ ID NO: 10 - DNA, 89 bp, Artificial Sequence
  Synthetic oligonucleotide
tgcacgaaggtcctactgtggaaaaaaaagggacaggattaagatattgatagttagtaatgacgcgtcgcgacgatcggaccacttac

SEQ ID NO: 11 - DNA, 27 bp, Artificial Sequence
  Synthetic oligonucleotide
ctcgagcatggtgagcggcctgctgaa

SEQ ID NO: 12 - DNA, 27 bp, Artificial Sequence
  Synthetic oligonucleotide
tctagaagttggccttctcgggcaggt

SEQ ID NO: 13 - DNA, 91 bp, Artificial Sequence
  Synthetic oligonucleotide
cctggccccctgctgcgagtacggcagcaggacgtaagtggatccgatcgtcccaccacttaaacgtggatgtacttgctttgaaactaaa

SEQ ID NO: 14 - DNA, 20 bp, Artificial Sequence
  Synthetic oligonucleotide
gaggagttgagggtactgtg

SEQ ID NO: 15 - DNA, 20 bp, Artificial Sequence
  Synthetic oligonucleotide
gaggagctgagggcactgtg

SEQ ID NO: 16 - DNA, 21 bp, Artificial Sequence
  Synthetic oligonucleotide
gtagaagtgcctctgccttcc

SEQ ID NO: 17 - DNA, 27 bp, Artificial Sequence
  Synthetic oligonucleotide
gaggctggagtagaaggattgctttgg

SEQ ID NO: 18 - DNA, 27 bp, Artificial Sequence
  Synthetic oligonucleotide
ccctcctgacccatcacctccaccacc

SEQ ID NO: 19 - DNA, 24 bp, Artificial Sequence
  Synthetic oligonucleotide
nnnnnnnnnnnnnnnnnnnnnnnn

SEQ ID NO: 20 - DNA, 40 bp, Artificial Sequence
  Synthetic oligonucleotide
ggccagtgaattgtaatacgactcactatagggaggcggn